(19) United States
(12) Patent Application Publication
(10) Pub. No.: US 2023/0404386 A1
(43) Pub. Date: Dec. 21, 2023

(54) INTERNAL CALIBRATION FOR AUTO-PHOROPTER

(71) Applicant: iCRx, Inc., Tucson, AZ (US)

(72) Inventors: Erol Ozgur, Tucson, AZ (US); Pierre A. Blanche, Tucson, AZ (US)

(21) Appl. No.: 17/843,724

(22) Filed: Jun. 17, 2022

(51) Int. Cl. A61B 3/028; A61B 3/10; A61B 3/04

(52) U.S. Cl. CPC A61B 3/0285; A61B 3/1015; A61B 3/04; A61B 2560/0223

(57) ABSTRACT

The present invention is directed to an automated ophthalmic aberration measurement by an auto-phoropter. In some embodiments, the present invention features a vision testing system capable of automated calibration. In some embodiments, the system may comprise a phoropter capable of measuring the ophthalmic aberration of an eye, and providing the necessary correction. The phoropter may comprise a wavefront sensor, one or more lenses calibrated using an initial correlation factor, a model eye disposed within the phoropter for internal calibration, and a light redirection component disposed within the phoropter. The light redirection component may be capable of redirecting light into the model eye to determine an optimal correlation factor.

### FIELD OF THE INVENTION

The present invention is directed to methods and devices for calibration of phoropters configured for automated ophthalmic aberration measurement.

### BACKGROUND OF THE INVENTION

As defined for the purpose of this invention, an auto-phoropter is an instrument capable of measuring the ophthalmic aberration of an eye, and providing the necessary correction so the user can appreciate the proposed correction and judge the restored accuity. The auto-phoropter combines the function of an auto-refractometer, that measures the eye aberration, and a phoropter, that corrects vision.

The auto-phoropter uses the wavefront measurement of the light reflected by the retina of the eye to determine the ophthalmic aberration, and then applies this correction to restore the vision acuity. It is therefore apparent that a correlation must be established between the measurement and the correction applied. This correlation can be applied in the factory when the instrument is built. However, there is a risk that the correlation factor could change over time due to external or internal factors such as temperature, humidity, system misalignment during transport, or else.

When the correlation factor changes the system must be re-calibrated. Usually, this means the system needs to be shipped back to the factory, or at a minimum, needs to have a specialized technician come and service the instrument. Thus, there exists a present need for a method that allows for automated calibration of auto-phoropters.

### BRIEF SUMMARY OF THE INVENTION

It is an objective of the present invention to provide systems and methods that allow for internal calibration of an auto-phoropter device, as specified in the independent claims. Embodiments of the invention are given in the dependent claims. Embodiments of the present invention can be freely combined with each other if they are not mutually exclusive.

The present invention features a vision testing system capable of automated internal calibration. In some embodiments, the system may comprise an automatic phoropter capable of measuring the ophthalmic aberration of an eye, and providing the necessary correction. In some embodiments, the phoropter component may comprise a wavefront sensor configured to measure the ophthalmic aberration of the eye, lenses configured to correct the measured ophthalmic aberration, an internal model eye disposed within the phoropter for internal calibration, a first optical path between the wavefront sensor and a test position for the eye, a second optical path between the wavefront sensor and the internal model eye, and a light redirection component configured to selectively enable either testing via the first optical path or calibration via the second optical path. The system may further comprise a computing device operatively coupled to the phoropter. The computing device may comprise a processor and a memory component comprising a plurality of computer-readable instructions for accepting a recalibration request, enabling the second optical path, measuring an ophthalmic aberration of the model eye via the wavefront sensor, and determining an optimal correlation factor based on a difference between the measured aberration of the model eye and a known value.

In some embodiments, the present invention features a vision testing system capable of automated calibration. In some embodiments, the system may comprise a phoropter capable of measuring the ophthalmic aberration of an eye, and providing the necessary correction. The phoropter may comprise a wavefront sensor, one or more lenses calibrated using an initial correlation factor, a model eye disposed within the phoropter for internal calibration, and a light redirection component disposed within the phoropter. The light redirection component may be capable of redirecting light into the model eye to determine an optimal correlation factor.

The present invention features a method for automated internal calibration of a vision testing system. In some embodiments, the method may comprise providing an automatic phoropter capable of measuring the ophthalmic aberration of an eye, and providing the necessary correction. In some embodiments, the phoropter component may comprise a wavefront sensor configured to measure the ophthalmic aberration of the eye, one or more lenses configured to correct the measured ophthalmic aberration, an internal model eye disposed within the phoropter for internal calibration, a first optical path between the wavefront sensor and a test position for the eye, a second optical path between the wavefront sensor and the internal model eye, and a light redirection component configured to selectively enable either testing via the first optical path or calibration via the second optical path. The method may further comprise accepting a recalibration request, enabling the optical path, measuring an ophthalmic aberration of the model eye via the wavefront sensor, and determining an optimal correlation factor based on a difference between the measured aberration of the model eye and a known value.

One of the unique and inventive technical features of the present invention is the implementation of an internal model eye for calibrating a phoropter device. Without wishing to limit the invention to any theory or mechanism, it is believed that the technical feature of the present invention advantageously provides for efficient and consistent calibration of a phoropter device in response to temperature/pressure changes or by request of a technician without the need for external components. None of the presently known prior references or work has the unique inventive technical feature of the present invention.

Furthermore, the prior references teach away from the present invention. For example, prior systems teach manual calibration of phoropter systems by a specialist to control the effect of thermal expansion on the adaptive optical components and ensure that everything is in order for the next patient measurement. On the contrary, the present invention is able to implement automatic calibration of phoropter systems through use of a model eye and a specialized technique for controlling the effect of thermal expansion on the adaptive optical components. This is able to calibrate a phoropter system automatically with accuracy on par or exceeding that of manual calibration, and without requiring a specialist to double-check, thus achieving greater time-efficiency. Thus, the prior references teach away from the presently claimed invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1 shows a schematic of the system for internal phoropter calibration of the present invention.

FIG. 2 shows a flow chart of a method for internal phoropter calibration of the present invention.

FIG. 3 shows an exemplary embodiment of the model eye of the present invention comprising a quarter wave plate and a mirror.

### DETAILED DESCRIPTION OF THE INVENTION

Following is a list of elements corresponding to a particular element referred to herein:
100 vision testing system
110 automatic phoropter
111 wavefront sensor
112 lens
113 model eye
114 first optical path
115 second optical path
116 light redirection component

The present invention features a procedure that allows for an auto-phoropter can be re-calibrated internally without the need to open the instrument, ship it back to the factory, use external device, or even to require special training.

The calibration of an auto-phoropter is usually done by placing a model eye in front of the instrument objective. The model eye replaces the eye of a subject, and has a known refractive power that is used to calibrate the instrument.

Instead of having an external model eye that needed to be manually placed in front of the instrument, it was found to be more convenient to have the model eye placed inside the instrument and to use a mechanism to redirect the light to the model eye when the system needs to be re-calibrated.

The mechanism to redirect the light can be of various nature such as a rotating polarizer together with a polarizing beam splitter, an electro-optics cell with a polarizing beam splitter, a mobile mirror mounted on an actuator, a digital micro-mirror device, and more.

The mechanism to redirect the light to the model eye can be activated at regular time intervals, before every measurement, when sensors sense atmospheric variation (temperature and/or pressure), or at the request of an operator.

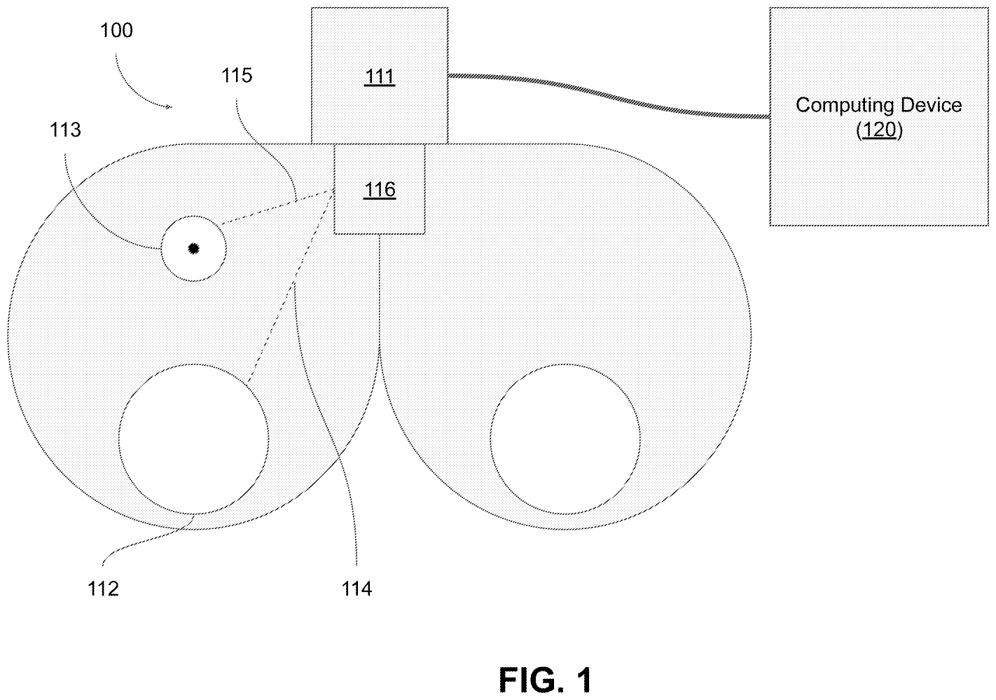

Referring now to FIG. 1, the present invention features a vision testing system (100) capable of automated internal calibration. In some embodiments, the system (100) may comprise an automatic phoropter (110) capable of measuring the ophthalmic aberration of an eye, and providing the necessary correction. In some embodiments, the phoropter component (110) may comprise a wavefront sensor (111) configured to measure the ophthalmic aberration of the eye, one or more lenses (112) configured to correct the measured ophthalmic aberration, an internal model eye (113) disposed within the phoropter (110) for internal calibration, a first optical path (114) between the wavefront sensor (111) and a test position for the eye, a second optical path (115) between the wavefront sensor (111) and the internal model eye (113), and a light redirection component (116) configured to selectively enable either testing via the first optical path (114) or calibration via the second optical path (115).

The system (100) may further comprise a computing device (120) operatively coupled to the phoropter (110). The computing device (120) may comprise a processor capable of executing computer-readable instructions, and a memory component comprising a plurality of computer-readable instructions. The plurality of computer-readable instructions may comprise accepting a recalibration request, enabling the second optical path (115), measuring an ophthalmic aberration of the model eye (113) via the wavefront sensor (111), and determining an optimal correlation factor based on a difference between the measured aberration of the model eye (113) and a known value. In some embodiments, the memory component may further comprise computer-readable instructions for determining an optimal correction factor based on a difference between the measured aberration of the model eye (113) and a known correction value. The optimal correction may be calculated by measuring, by the wavefront sensor (111), a wavefront error of light reflected from the model eye (113)

In some embodiments, the light redirection component (116) may comprise a rotating polarizer and a polarizing beam splitter. In other embodiments, the light redirection component (116) may comprise an electro-optics cell and a polarizing beam splitter. In other embodiments, the light redirection component (116) may comprise a mobile mirror mounted on an actuator. The system (100) may further comprise one or more atmospheric sensors communicatively coupled to the computing device (120). Each atmospheric sensor may be capable of measuring environmental temperature, environmental pressure, or a combination thereof. Accordingly, the memory component may further comprise computer-readable instructions for detecting, by the one or more atmospheric sensors, a change in environmental temperature, environmental pressure, or a combination thereof, and transmitting a recalibration request in response to the change in environmental temperature, environmental pressure, or a combination thereof.

In some embodiments, the present invention features a vision testing system (100) capable of automated calibration for adaptive optical components (e.g. fluidic lens). In some embodiments, the system (100) may comprise a phoropter (110) capable of measuring the ophthalmic aberration of an eye, and providing the necessary correction. The phoropter (110) may comprise a wavefront sensor (111), one or more lenses (112) calibrated using an initial correlation factor, a model eye (113) disposed within the phoropter (110) for internal calibration, and a light redirection component (116) disposed within the phoropter (110). The light redirection component (116) may be capable of redirecting light into the model eye (113) to determine an optimal correlation factor.

The phoropter (110) may be actuated by receiving a recalibration request from an external source. In some embodiments, the external source may comprise a computing device (120). In other embodiments, the external source may comprise one or more atmospheric sensors capable of detecting a change in temperature, pressure, or a combination thereof. In some embodiments, the light redirection component (116) may comprise a rotating polarizer and a polarizing beam splitter. In other embodiments, the light redirection component (116) may comprise an electro-optics cell and a polarizing beam splitter. In other embodiments, the light redirection component (116) may comprise a mobile mirror mounted on an actuator.

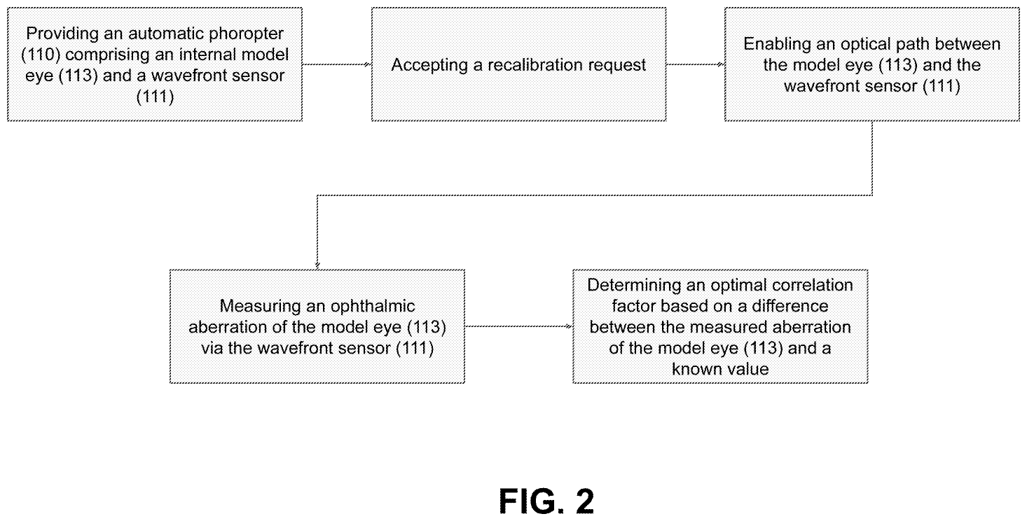

Referring now to FIG. 2, the present invention features a method for automated internal calibration for the adaptive optical components (e.g. fluidic lens) of a vision testing system. In some embodiments, the method may comprise providing an automatic phoropter (110) capable of measuring the ophthalmic aberration of an eye, and providing the necessary correction. In some embodiments, the phoropter component (110) may comprise a wavefront sensor (111) configured to measure the ophthalmic aberration of the eye, one or more lenses (112) configured to correct the measured ophthalmic aberration, an internal model eye (113) disposed within the phoropter (110) for internal calibration, a first optical path (114) between the wavefront sensor (111) and a test position for the eye, a second optical path (115) between the wavefront sensor (111) and the internal model eye (113), and a light redirection component (116) configured to selectively enable either testing via the first optical path (114) or calibration via the second optical path (115). The method may further comprise accepting a recalibration request, enabling the optical path, measuring an ophthalmic aberration of the model eye (113) via the wavefront sensor (111), and determining an optimal correlation factor based on a difference between the measured aberration of the model eye (113) and a known value. In some embodiments, the method may further comprise determining an optimal correction factor based on a difference between the measured aberration of the model eye (113) and a known correction value. The optimal correction may be calculated by measuring, by the wavefront sensor (111), a wavefront error of light reflected from the model eye (113)

In some embodiments, the light redirection component (116) may comprise a rotating polarizer and a polarizing beam splitter. In other embodiments, the light redirection component (116) may comprise an electro-optics cell and a polarizing beam splitter. In other embodiments, the light redirection component (116) may comprise a mobile mirror mounted on an actuator. In some embodiments, the method may further comprise providing one or more atmospheric sensors communicatively coupled to the computing device (120). Each atmospheric sensor may be capable of measuring environmental temperature, environmental pressure, or a combination thereof. The method may further comprise detecting, by the one or more atmospheric sensors, a change in environmental temperature, environmental pressure, or a combination thereof, and transmitting a recalibration request in response to the change in environmental temperature, environmental pressure, or a combination thereof.

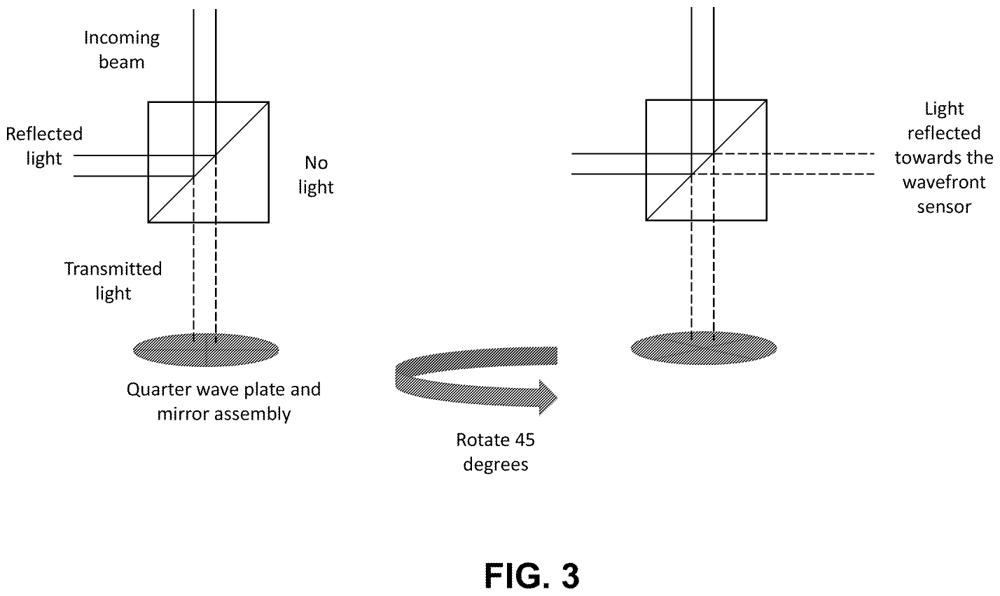

In some embodiments, the model eye may comprise a quarter wave plate and a mirror. The rotation of the quarter wave plate may determine whether the light will be reflected towards the sensor or not. This way, a more compact system with no additional optical paths can be achieved. This is depicted in FIG. 3.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A vision testing system (100) capable of automated internal calibration comprising:
a. an automatic phoropter (110) capable of measuring the ophthalmic aberration of an eye, and providing the necessary correction, the phoropter component (110) comprising:
i. a wavefront sensor (111) configured to measure the ophthalmic aberration of the eye;
ii. one or more lenses (112) configured to correct the measured ophthalmic aberration;
iii. an internal model eye (113) disposed within the phoropter (110) for internal calibration;
iv. a first optical path (114) between the wavefront sensor (111) and a test position for the eye;
v. a second optical path (115) between the wavefront sensor (111) and the internal model eye (113); and
vi. a light redirection component (116) configured to selectively enable either testing via the first optical path (114) or calibration via the second optical path (115); and
b. a computing device (120) operatively coupled to the phoropter (110), comprising a processor capable of executing computer-readable instructions, and a memory component comprising a plurality of computer-readable instructions for:
i. accepting a recalibration request;
ii. enabling the second optical path (115);
iii. measuring an ophthalmic aberration of the model eye (113) via the wavefront sensor (111); and
iv. determining an optimal correlation factor based on a difference between the measured aberration of the model eye (113) and a known value.

2. The system (100) of claim 1, wherein the memory component further comprises computer-readable instructions for:
a. determining an optimal correction factor based on a difference between the measured aberration of the model eye (113) and a known correction value;
wherein the optimal correction is calculated by measuring, by the wavefront sensor (111), a wavefront error of light reflected from the model eye (113).

3. The system (100) of claim 1, wherein the light redirection component (116) comprises a rotating polarizer and a polarizing beam splitter.

4. The system (100) of claim 1, wherein the light redirection component (116) comprises an electro-optics cell and a polarizing beam splitter.

5. The system (100) of claim 1, wherein the light redirection component (116) comprises a mobile mirror mounted on an actuator.

6. The system (100) of claim 1 further comprising one or more atmospheric sensors communicatively coupled to the computing device (120), wherein each atmospheric sensor is capable of measuring environmental temperature, environmental pressure, or a combination thereof, wherein the memory component further comprises computer-readable instructions for:
a. detecting, by the one or more atmospheric sensors, a change in environmental temperature, environmental pressure, or a combination thereof; and
b. transmitting a recalibration request in response to the change in environmental temperature, environmental pressure, or a combination thereof.

7. A vision testing system (100) capable of automated calibration comprising:
a. a phoropter (110) capable of measuring the ophthalmic aberration of an eye, and providing the necessary correction, the phoropter (110) comprising:
i. a wavefront sensor (111);
ii. one or more lenses (112) calibrated using an initial correlation factor;
iii. a model eye (113) disposed within the phoropter (110) for internal calibration; and
iv. a light redirection component (116) disposed within the phoropter (110);
wherein the light redirection component (116) is capable of redirecting light into the model eye (113) to determine an optimal correlation factor.

8. The system (100) of claim 7, wherein the phoropter (110) is actuated by receiving a recalibration request from an external source.

9. The system (100) of claim 8, wherein the external source comprises a computing device (120).

10. The system (100) of claim 8, wherein the external source comprises one or more atmospheric sensors capable of detecting a change in temperature, pressure, or a combination thereof.

11. The system (100) of claim 7, wherein the light redirection component (116) comprises a rotating polarizer and a polarizing beam splitter.

12. The system (100) of claim 7, wherein the light redirection component (116) comprises an electro-optics cell and a polarizing beam splitter.

13. The system (100) of claim 7, wherein the light redirection component (116) comprises a mobile mirror mounted on an actuator.

14. A method for automated internal calibration of a vision testing system, the method comprising:
a. providing an automatic phoropter (110) capable of measuring the ophthalmic aberration of an eye, and providing the necessary correction, the phoropter component (110) comprising:
i. a wavefront sensor (111) configured to measure the ophthalmic aberration of the eye;
ii. one or more lenses (112) configured to correct the measured ophthalmic aberration;
iii. an internal model eye (113) disposed within the phoropter (110) for internal calibration;
iv. a first optical path (114) between the wavefront sensor (111) and a test position for the eye;
v. a second optical path (115) between the wavefront sensor (111) and the internal model eye (113); and
vi. a light redirection component (116) configured to selectively enable either testing via the first optical path (114) or calibration via the second optical path (115);
b. accepting a recalibration request;
c. enabling the optical path;
d. measuring an ophthalmic aberration of the model eye (113) via the wavefront sensor (111); and
e. determining an optimal correlation factor based on a difference between the measured aberration of the model eye (113) and a known value.

15. The method of claim 13 further comprising:
a. determining an optimal correction factor based on a difference between the measured aberration of the model eye (113) and a known correction value.

16. The method of claim 13, wherein the light redirection component (116) comprises a rotating polarizer and a polarizing beam splitter.

17. The method of claim 13, wherein the light redirection component (116) comprises an electro-optics cell and a polarizing beam splitter.

18. The method of claim 13, wherein the light redirection component (116) comprises a mobile mirror mounted on an actuator.

19. The method of claim 13 further comprising:
a. providing one or more atmospheric sensors communicatively coupled to the computing device (120), wherein each atmospheric sensor is capable of measuring environmental temperature, environmental pressure, or a combination thereof;
b. detecting, by the one or more atmospheric sensors, a change in environmental temperature, environmental pressure, or a combination thereof; and
c. transmitting a recalibration request in response to the change in environmental temperature, environmental pressure, or a combination thereof.
